(19) United States
(12) Patent Application Publication
(10) Pub. No.: US 2007/0072166 A1
(43) Pub. Date: Mar. 29, 2007

(54) Semen preservation

(76) Inventors: Anthony Michael, London (GB); Lisa Thurston, London (GB)

Correspondence Address: NIXON & VANDERHYE, PC, 901 NORTH GLEBE ROAD, 11TH FLOOR, ARLINGTON, VA, 22203, US

(21) Appl. No.: 10/578,559

(22) PCT Filed: Nov. 8, 2004
(86) PCT No.: PCT/GB04/04683

§ 371 Date: Sep. 14, 2006

(30) Foreign Application Priority Data
Nov. 6, 2003 (GB) 0325968.6

(51) Int. Cl. A01N 1/02; C12Q 1/00

(52) U.S. Cl. 435/2; 435/4

(57) ABSTRACT

The present invention provides a method for determining the suitability of a sample of mammalian semen for cooling and/or cryopreservation or storage, which comprises: (a) providing said sample of semen; (b) determining the level of a hydrophobic stimulator of 11β-HSD activity in said sample; and (c) assessing, from the level of 11β-HSD stimulator determined, the suitability of the semen sample for cooling and/or cryopreservation or storage. The present invention also provides a method of obtaining a hydrophobic product that improves the tolerance of mammalian semen to cooling and/or cryopreservation or storage, a method of improving the survival rate of sperm intended for cooling and/or cryopreservation or storage and a method of performing an assisted conception/reproductive procedure comprising contacting an oocyte with sperm obtained by a method according to the present invention.

### FIELD OF THE INVENTION

This invention relates to modulators of the enzyme 11β-hydroxysteroid dehydrogenase (11β-HSD) present in mammalian semen. More specifically, the invention relates to the use of such modulators in improving the viability of cooled and/or cryopreserved or stored sperm and to predicting the suitability of a semen sample for cooling and/or cryopreservation or storage, with a view to later use in assisted conception/reproductive techniques, in humans or livestock mammals. It also relates to means for improving the suitability of semen samples for use in such techniques.

### BACKGROUND OF THE INVENTION

The techniques of cooling and cryopreservation of semen are used to store semen at temperatures used for assisted conception/reproductive techniques. There are, however, difficulties associated with such techniques. In particular, when cooled or frozen semen samples are raised back to temperatures used in assisted conception/reproductive techniques, the sperm that they contain may become non-viable. The enzyme 11β-HSD converts cortisol to its inactive form, cortisone. Nacharaju et al (1997) have noted the presence of 11β-HSD in human semen and suggest a role for 11β-HSD in maintaining sperm development and function.

Previous work has shown that 11β-HSD activity in female follicular fluid is involved in determining the success of the outcome of assisted conception/reproductive techniques. Generally, lower levels of 11β-HSD in follicular fluid are desirable to achieve successful assisted conception/reproductive treatment (WO 94/21815). It has also been shown that there are modulators of 11β-HSD activity in female follicular fluid which influence the activity and hence effects of 11β-HSD (WO 97/30175 and Thurston et al (2002a)). These modulators have been characterised to a certain extent but not identified in chemical terms. The modulators were found to elute into two fractions on a C18 column (Thurston et al (2002a)). The first modulator fraction eluted at from 0 to 20% methanol. It is hydrophilic and stimulates 11β-HSD activity. The presence of the stimulatory modulator(s) in female follicular fluid was found to decrease the chances of successful assisted conception/reproductive techniques (Thurston et al (2003a)). The second modulator fraction elutes at from 60 to 100% methanol. It is hydrophobic and inhibits 11β-HSD activity. The presence of the inhibitory modulator(s) in female follicular fluid was found to increase the chances of successful assisted conception/reproductive techniques.

### SUMMARY OF THE INVENTION

We have found two new groups of modulators of 11β-HSD activity in porcine (boar) semen. These protect sperm from death induced by cooling and cryopreservation. They also improve the viability of sperm stored at room temperature. The two modulator fractions elute in two different fractions on a C18 column. The first is from 50 to 75% methanol. The second is from 95 to 100% methanol. Both groups of modulators are therefore hydrophobic in nature. Both stimulate 11β-HSD activity in rat kidney homogenates.

More specifically, we found that modulators of 11β-HSD activity in porcine (boar) semen which elute from a C18 column at 65% and 100% (v/v) methanol protect sperm from death after full cryopreservation and thawing.

We have also identified hydrophobic modulatory fractions in bovine (bull) semen.

We found that, when semen samples are cooled and rewarmed and/or cryopreserved and thawed, those with high modulator levels exhibited higher levels of sperm viability. These findings are surprising based on what was known in the art. Firstly, it was known (WO 94/21815, and see also Michael et al (1993) and Michael et al (1995)) that low levels of 11β-HSD in the environment of the oocyte increased the chances of successful assisted conception/reproductive techniques in particular in vitro fertilization (IVF). However, we have now found that high levels of 11β-HSD stimulator (as isolated from seminal plasma) in semen are preferred in the context of preserving semen for assisted conception/reproductive techniques. Secondly, the modulators identified in female follicular fluid elute into two bands: hydrophobic 11β-HSD inhibitors and hydrophilic 11β-HSD stimulators. In contrast, the current invention has shown the presence of two hydrophobic modulators in semen both of which stimulate 11β-HSD activity. We did not find semen containing inhibitors, whether hydrophobic or hydrophilic.

A disadvantage of assisted conception/reproductive techniques which utilize cooled and/or cryopreserved semen is that the sperm often tend to lose their viability and/or longevity when the semen sample is brought back up to a temperature suitable for assisted conception/reproductive techniques.

The present invention thus relates to methods of determining the presence of the new modulators of 11β-HSD activity in semen and thus the suitability of such semen for cooling and/or cryopreservation.

The invention also relates to a method of improving the viability of cooled or cryopreserved sperm. By providing a more optimal concentration of the new 11β-HSD modulators it will be possible to recover a greater proportion of viable sperm after cooling and/or cryopreservation. Due to the presence of the modulators of the present invention, the resuspended semen sample is more likely to be viable for use in assisted conception/reproductive techniques.

In humans, assisted conception/reproductive techniques are expensive procedures and can be psychologically traumatic for patients. Therefore, until success rates are improved it would be desirable to be able to identify those men who produce semen likely to remain viable after cooling and/or cryopreservation by virtue of the high concentration of the modulators of the present invention. This will enable couples in which the male partner has unsuitable semen to choose to avoid assisted conception/reproductive techniques, if they wish, and also to avoid the banking of unsuitable semen for anonymous use. It will also make it possible to screen the seminal plasma of men recruited by assisted conception clinics for semen donation. Similarly, it will be possible to help individuals with unsuitable semen by providing more optimal concentrations of modulators.

An advantage of the current invention is therefore to facilitate a good recovery of viable sperm following cooling or cryopreservation, and so to increase the chances of successful assisted conception/reproductive techniques with the warmed sperm.

Additionally, in the context of livestock, certain mammalian species' semen samples, for example pig and horse semen, are expensive, and some species' semen is more prone than that of others to losing viability when cooled/cryopreserved. The current invention is therefore economically advantageous in that it reduces costs by improving the chances of successful assisted conception/reproductive techniques, in livestock. It may also increase the number of offspring that can be obtained from particular high quality individuals that unfortunately happen to have sperm susceptible to a high rate of cooling-induced cell death.

The present invention will be applicable to any mammalian male and can be used not only in human assisted conception/reproductive programmes but also to increase the success of, for example, captive breeding programmes of endangered species or commercial breeding by assisted conception/reproductive techniques of livestock such as cattle, pigs and horses. It will also be applicable to rodent sperm, which will be of particular value in cooling/cryopreservation or storage of the sperm of transgenic mice.

Accordingly, the invention provides a method of determining the suitability of a sample of mammalian semen for cooling and/or cryopreservation or storage, said method comprising:
(a) providing said sample of semen;
(b) determining the level of a hydrophobic stimulator of 11β-HSD activity in said sample; and
(c) assessing, from the level of 11β-HSD stimulator determined, the suitability of the semen sample for cooling and/or cryopreservation or storage.

The invention also provides a method of improving the survival rate of sperm or promoting the viability of sperm, said method comprising:
(a) providing a sample of semen; and
(b) combining said sample of semen with an increased concentration of a hydrophobic stimulator of 11β-HSD activity; and optionally
(c) storing said combination of semen and hydrophobic stimulator for a period of time.

The invention also provides a method of fertilizing an oocyte in vitro comprising contacting said oocyte with sperm obtained by a method of the invention, said method comprising:
(a) providing a sample of semen; and
(b) combining said sample of semen with an increased concentration of a hydrophobic stimulator of 11β-HSD activity under conditions which allow fertilization of the oocyte.

The invention also provides a method of performing an assisted conception/reproductive procedure comprising contacting an oocyte with sperm obtained by a method of the invention, said method comprising:
(a) providing a sample of semen; and
(b) combining said sample of semen with an increased concentration of a hydrophobic stimulator of 11β-HSD activity under conditions which allow fertilization of the oocyte.

The invention also provides a method of obtaining a hydrophobic product that improves the tolerance of mammalian semen to cooling and/or cryopreservation or storage, comprising the steps of:
(a) providing a sample of semen;
(b) removing the seminal plasma from the sperm; and
(c) fractionating the seminal plasma of (b) to enrich for said product.

The invention also provides a product obtainable by fractionation of mammalian seminal plasma and having a stimulatory effect on 11β-HSD activity, which improves the tolerance of semen to cooling and/or cryopreservation or storage.

The invention also provides the use of said product to improve the tolerance semen to cooling and/or cryopreservation or storage.

The invention also provides the use of said product in the manufacture of a medicament for use in the treatment of inflammatory disease by increasing the survival of topically applied cortisol or cortisol already circulating within the bloodstream.

The invention also provides the use of said product in the manufacture of a medicament for use in the treatment of inflammatory disease by stimulating the production of cortisol from circulating cortisone by stimulation of 11β-HSD1.

### BRIEF DESCRIPTION OF THE DRAWINGS

FIG. 1: Effects of fractions of seminal plasma eluted from a C18 column with different concentrations of methanol on 11β-HSD activity.

FIG. 2: Variation in the effect of lipid extracted from seminal plasma on sperm viability following cooling to 5° C. and rewarming to 39° C. (mean+/−SEM; n=5; values with different superscripts differ −P<0.05). SP1, SP2 and SP3 denote effects of seminal plasma samples taken from three separate male boars.

FIG. 3: Effects of fractions of seminal plasma elated from a C18 column with different concentrations of methanol on the survival of sperm following full cryopreservation and thawing.

FIG. 4: Effects of C18 fractions eluted from 10 bovine seminal plasma samples on 11βHSD1 activity (NADP-dependent oxidation of cortisol) in rat kidney homogenates. Each data point is the mean ±SEM of values for seminal plasma from 10 independent bulls. The horizontal dashed line indicates the control enzyme activity, set to 100%. *P<0.05 and **P<0.01 versus respective control enzyme activity. (Data subjected to one-way ANOVA+Durnnett's multiple comparison).

FIG. 5: Effects of C18 fractions of nine porcine seminal plasma samples on 11βHSDL activity (NADP-dependent oxidation of cortisol) in a rat kidney homogenate. Each data point is the mean±SEM of values for seminal plasma from 8 independent boars. The horizontal dashed line indicates the control enzyme activity, set to 100%. *P<0.05 and **P<0.0l versus respective control enzyme activity. (Data subjected to one-way ANOVA+Dunnett's multiple comparison).

FIG. 6: Correlation between the level of stimulation of 11βHSD1 activity (NADP-dependent oxidation of cortisol) exerted by the major hydrophobic fraction of bull seminal plasma (eluted at 55% v/v methanol) and (A) the proportion of live sperm, and (B) the proportion of osmotic resistant sperm in the corresponding ejaculates.

FIG. 7: Correlation between the level of stimulation of 11βHSD1 activity (NADP-dependent oxidation of cortisol) exerted by the major hydrophobic fraction of boar seminal plasma (eluted at 65% v/v methanol) and (A) the proportion of live sperm, and (B) the proportion of osmotic resistant sperm in the corresponding ejaculates.

FIG. 8: Effects of C18 fractions of bovine seminal plasma sample on survival of bull sperm for 3 days at room temperature in a commercial extender solution.

Each data point is the mean±SEM of values for sperm from 3 independent bulls. The horizontal dashed line indicates the viability of sperm maintained in the absence of seminal plasma fractions. (No column fractions exerted any significant effect on bull sperm survival at room temperature: ANOVA P=0.198).

FIG. 9: Effects of C18 fractions of porcine seminal plasma on survival of boar sperm for 3 days at room temperature in a commercial extender solution. Each data point is the mean±SEM of values for sperm from 5 independent boars. The horizontal dashed line indicates the viability of sperm maintained in the absence of seminal plasma fractions. (Data subjected to one-way ANOVA+Dunnett's multiple comparison).

FIG. 10: Effects of C18 fractions of porcine seminal plasma on motility of boar sperm after 3 days storage at room temperature in a commercial extender solution. Each data point is the mean±SEM of values for sperm from 3 independent boars. The horizontal dashed line indicates the motility of sperm maintained in the absence of seminal plasma fractions. (No column fractions exerted any significant effect on boar motility at room temperature: ANOVA; P=0.401).

FIG. 11: Correlation between the level of stimulation of 11βHSD1 activity (NADP-dependent oxidation of cortisol) exerted by the major hydrophobic fraction of bull seminal plasma (eluted at 55% v/v methanol) and (A) the proportion of live sperm, (B) the proportion of osmotic resistant sperm, and (C) the proportion of acrosome intact sperm in the corresponding ejaculates following freezing and thawing.

FIG. 12: Correlation between the level of stimulation of 11βHSD1 activity (NADP-dependent oxidation of cortisol) exerted by the major hydrophobic fraction of boar seminal plasma (eluted at 65% v/v methanol) and (A) the proportion of live sperm, (1) the proportion of osmotic resistant sperm, and (C) the proportion of acrosome intact sperm in the corresponding ejaculates following freezing and thawing.

FIG. 13: Effects of C18 fractions of porcine seminal plasma on viability of boar sperm after cryopreservation in a commercial cryoprotectant solution. Each data point is the mean±SEM of values for sperm from 3 independent boars. The horizontal dashed line indicates the viability of sperm cryopreserved in the absence of seminal plasma fractions. (Data subjected to one-way ANOVA+Dunnett's multiple comparison).

FIG. 14: Effects of C18 fractions of porcine seminal plasma on proportion of motile boar sperm after cryopreservation in a commercial cryoprotectant solution. Each data point is the mean±SEM of values for sperm from 3 independent boars. The horizontal dashed line indicates the motility of sperm cryopreserved in the absence of seminal plasma fractions. (Data subjected to one-way ANOVA+Dunnett's multiple comparison).

### DETAILED DESCRIPTION OF THE INVENTION

The present invention provides a method of determining the suitability of a sample of semen for cooling and/or cryopreservation or storage. The invention also provides a method of improving the survival rate of sperm, or promoting the viability of sperm, intended for cooling and/or cryopreservation or storage. The invention further provides a method of obtaining a hydrophobic product that improves the tolerance of semen to cooling and/or cryopreservation or storage.

Obtaining Samples of Semen

The first step in the methods of the current invention is obtaining a semen sample. Semen from any species can be selected and assayed for the presence of a hydrophobic stimulator of 11β-HSD activity. Semen samples are preferably collected by manual ejaculation methods, through use of an artificial vagina (AV), or electro-ejaculation. The method can be carried out with sperm from any mammal, notably human sperm and that of domesticated animals, especially livestock animals, as well as with sperm from wild animals (e.g. endangered species). Human sperm, bovine, equine, porcine and ovine sperm are more preferred.

Sperm Viability

The techniques of the invention promote sperm viability. Herein, promoting viability typically means that sperm survival is promoted, i.e. that a greater promotion of sperms stay alive after cooling/cryopreservation or storage than would be the case in the absence of a technique of the invention. Preferably, sperm motility is also provided, i.e. a grater proportion of sperms remain mobile after cooling/cryopreservation or storage than would be the case in the absence of a technique of the invention. The proportion of acrosome intact sperm may also be increased relative to a situation in which a technique of the invention is not applied. Sperm motility and intactness of the acrosome are required for current IVF, Al and IUI techniques, though not for ICSI.

Determining the Level of Hydrophobic Stimulator of 11β-HSD Activity

The second step in the method of determining the suitability of a semen sample for cryopreservation and/or cooling or storage is to determine the level of hydrophobic stimulator of 11β-HSD activity in the sample of semen. By hydrophobic stimulator of 11β-HSD is meant a product that increases 11β-HSD activity. The product is hydrophobic in nature and preferably elutes from a C18 chromatography column at from 50 to 75% methanol or from 95 to 100% methanol. The hydrophobic stimulator may be an agonist or a cofactor of 11β-HSD activity.

Herein, a stimulatory fraction is typically hydrophobic if it elutes from a C18 chromatographic column at 40% methanol or more. In porcine (boar) semen, preferred hydrophobic stimulatory fractions elutes from 50 to 75% methanol preferably 55% to 70% methanol; and at 95% to 100% methanol, preferably 100% methanol. In bovine (bull) semen, preferred fractions elute at 55% and 60% methanol.

The elution range of hydrophobic stimulatory fractions may vary from species to species. However, hydrophobic stimulatory fractions may elute at from 40% to 100%, preferably 90% to 100%; 40% to 80%, preferably, 50% to 80%, more preferably 50% to 70%, 55% to 70%, 55% to 80%, 50% to 65%, 55% to 65%, 50% to 60%, 53% to 57%, 58% to 62%, 54% to 56% or 59% to 61%; or at 90% or greater, preferably 95% or greater, more preferably 99% or greater, e.g. 100%, methanol.

The level of hydrophobic stimulator of 11β-HSD activity may be measured directly (e.g. by determining the amount or concentration of the hydrophobic stimulator) or indirectly (by the level of 11β-HSD activity) as that will often be affected by the amount or concentration of hydrophobic stimulator present.

For example, the amount/concentration of hydrophobic stimulator may be measured through the conversion of a substrate of 11β-HSD (e.g. ³1-cortisol or ³H-corticosterone) to a product (e.g. ³H-cortisone or³H-11-dehyrocorticosterone respectively) in the absence and presence of hydrophobic stimulator. This involves contacting the sample with the substrate, for example ³H-cortisol, and measuring the conversion of the substrate to ³H-cortisone by 11β-HSD. Insuch a case the higher the level of cortisone in relation to cortisol, the higher the activity of the enzyme and hence higher the level of hydrophobic stimulator present in the sample. The level of ³H-cortisone and ³H-cortisol can be measured by methods known per se. TLC/HPLC may be used to resolve ³H-cortisol/³H-cortisone followed by the quantification of ³H-cortisol and ³H-cortisone levels by liquid scintillation counting or preferably through use of a TLC radiochromatogramme scanner. This method will provide a direct measurement of enzyme activity, and for this reason is preferred. In a typical assay a concentration range of about 100 nM of ³H-cortisol maybe used, although a concentration ranging from 10 nM to 1000 nM or more can be used.

Alternatively, or in addition, a semen sample may be contacted with 11β-HSD present in another body fluid or cultured cells or another body derived substance for example homogenised animal organs (such as rat kidney). A suitable control assay may also need to be conducted to allow for 11β-HSD already and naturally present in the sample. The 11β-HSD used in the assay may be from an isolated or purified source or can be present in another human or anrimal body or body derived fluid for example kidney homogenates. The 11β-HSD used in the assay may not necessarily be human. It may for example be of an anirnal source such as from a rodent. This can allow assays to be formed using relatively cheap and accessible forms of 11β-HSD e.g. rat kidney homogenates.

Alternatively 11β-HSD activity can be measured by immunoassay or similar ligand binding techniques. This will provide an indication of the amount of the enzyme, which may be correlated to enzyme activity and from there to the amount/concentration of hydrophobic stimulator of 11β-HSD activity. For example a ligand (or antibody) capable of binding the enzyme could be used in immunoassay methods such as RIA or ELISA. In addition, the level of 11β-HSD enzyme or even its mRNA can be used as a measure of a modulator activity since some modulators may exert their effects at the level of mRNA transcription or translation.

The expression of the 11β-HSD enzyme can also be measured by immunochemistry using a monoclonal antibody. Such techniques will provide a measurement of the amount of 11β-HSD present, which can then be correlated to enzyme activity.

Although reference is made in the specification to determining levels of 11β-HSD (and its modulators) it will be understood from the foregoing that this also includes the indirect measurements mentioned above.

The presence of these modulators in semen increases the viability of sperm following cryopreservation and/or cooling or storage, This is a surprising result, especially due to the well known fragility of sperm following cryopreservation and/or cooling. Researches have shown the stresses associated with cryopreservation resulted in losses in fertility and/or viability. The present inventors have identified modulators of 11β-HSD function which when present in semen samples increases the viability of semen undergoing cyopreservation and/or cooling, or storage.

Assessing the Suitability of the Semen Sample for Cooling and/or cryopreservation or Storage

Once the level of hydrophobic stimulator of 11β-HSD activity has been measured, the result can be used to determine the suitability of the semen sample for cryopreservation and/or cooling or storage. The level of 11β-HSD activity in the sample will be directly affected by the hydrophobic stimulator. Therefore, 11β-HSD activity will be proportional to the level of hydrophobic stimulator and a measurement of the level of hydrophobic stimulator can thus be correlated back to the level of 11β-HSD activity.

It is desirable to add to sperm an amount or concentration of 11β-HSD stimulator derived from seminal plasma which, when assessed at a dilution of 10% by volume, can increase 11β-HSD activity by 100% or more relative to enzyme activity measured in the absence of the stimulator.

The current invention has found that high levels of 11β-HSD stimulator in semen are preferred for successful assisted conception/reproductive techniques. In particular, those subjects who have hydrophobic stimulator present in their semen at a predetermined threshold indicated above, or exceeding this threshold are preferred as suitable candidates for having their semen cooled and/or cryopreserved.

For example, pig semen generally cools/freezes relatively poorly compared, say, to human sperm where typical survival rates might be, for example 70-80%. In pig semen samples with low levels of hydrophobic stimulator, only around 20 to 60% of sperm typically survive cooling and/or cryopreservation and rewarming. We have found that figure is significantly increased when the semen either contains or is supplemented with high level of hydrophobic stimulator. Semen having the hydrophobic stimulator at or above the above mentioned threshold is more likely to undergo cooling and/or cryopreservation or storage and still contain in excess of 70% viable sperm when the semen sample is brought back up to a temperature suitable for assisted conception/reproductive techniques.

Thus the amount of hydrophobic stimulator present in a sample of semen can be used to assess the likelihood of a semen sample to remain viable after cooling and/or cryopreservation or storage and hence whether such semen is likely to be viable for assisted conception/reproductive techniques. If the level of modulator is only determined for one ejaculate decisions as to the suitability of that particular male may only be possible based on the results for that ejaculate, although determinations over several ejaculated samples may give a more general indication of the suitability of the semen to undergo cooling and/or cryopreservation.

By use of the present invention, it will be possible for clinics which perform assisted conception/reproductive techniques, to allocate resources more efficiently. Male subjects with low levels of a hydrophobic stimulator of 11β-HSD activity in their semen, and thus have semen unsuitable for cooling and/or cryopreservation or storage, can be identified prior to freezing their semen, and the semen samples can b)e treated to promote therein viability.

Promoting the Viability Rate of Sperm Intended for Cooling and/or Cryopreservation

The current invention also provides methods of promoting the viability of sperm intended for cooling and/or cryopreservation. In one aspect of the present invention, a semen sample intended for cooling and cropreservation is obtained by methods known in the art or detailed above. The semen sample is then combined with an increased concentration of a hydrophobic stimulator of 11β-HSD activity.

It would be desirable to add to sperm an amount or concentration of 11β-HSD stimulator (derived from seminal plasma) which when assessed at a dilution of 10% by volume, can increase 11β-HSD activity by 100% or more relative to enzyme activity measured in the absence of the stimulator.

This method may additionally comprise an initial step of removing the desirable sperm from its seminal plasma followed by combining the desirable sperm with an increased concentration of hydrophobic stimulator of 11β-HSD. Sperm can be isolated from semen samples using any sufficiently gentle isolation method that provides at least about 50% recovery of sperm, preferably 75% or more preferably 90% recovery of sperm.

In a preferred embodiment, sperm separated from its seminal plasma may be combined with seminal plasma from another individual known to have an increased amount of hydrophobic stimulator of 11β-HSD activity in his seminal plasma Preferably this embodiment may apply to male animals other than humans.

The percentage sperm recovery using the centrifuge technique is approximately 90%. Percoll centifugation and Percoll ‘swim up’ may also be used to recover sperm cells. The percentage sperm recovered are approximately 75% and 50% for the Percoll centrifugation and Percoll ‘swim-up’ respectively.

The methods of the invention may be applied to any mammalian species. The semen may, for example, be of human origin, or of rodent, bovine, equine, porcine or ovine origin.

In the context of pig semen, the method of the invention preferably results in the survival of at least 40%, at least 50%, preferably at least 60% and more preferably at least 70% or more of the sperm following cooling and/or cryopreservation.

In the context of human semen, whose typical survival rate is higher (70-80%) in any case, the method of the invention preferably results in the survival of at least 85%, at least 90%, or at least 95% of the sperm following cooling and/or cryopreservation.

In the context of horse or sheep sperm, the method of the invention preferably results in the survival of at least 50%, at least 60% or at least 70% of the sperm following cooling and/or cryopreservation.

In the context of cow sperm, the method of the invention preferably results in the survival of at least 70%, at least 80% or at least 90% of the sperm following cooling and/or cryopreservation.

In the context of rodent (mouse or rat) sperm, the method of the invention preferably results in the survival of at least 60%, at least 70% or at least 80% of the sperm following cooling and/or cryopreservation.

In all species, the methods of the invention preferably result in sperm viability (e.g. survival) being increased significantly compared to a situation where the same individual's sperm is used, but without the benefit of the methods of the invention. The degree of improvement will vary from individual to individual within a species, and also from species to species. In species where sperm survival rates are already relatively high, e.g. in humans and cows the degree of improvement may be, say, at least 5%, at least 10%, at least 20%, at least 25% or at least 50%. However, in particular individuals with low sperm survival rates, the degree of improvement may be higher, e.g. 100% or more, or 200% or more. In species with relatively low sperm survival rates, for example pigs, sheep and horses, the degree of improvement may be, for example, at least 5%, at least 10%, at least 20%, at least 25%, at least 50%, at least 75% or at least 100% or more. In individuals with low sperm survival rates, the degree of improvement may be even higher, e.g. 200% or more.

Any of a variety of methods suitable for recovering cells from a suspension can be used to isolate the sperm, including for example filtration, sedimentation and centrifugation. In a preferred embodiment, the selected semen sample is aliquoted into 50 ml tubes at volumes not exceeding about 27 ml, and preferably from between about 20 to 27 ml. Centrifugation is carried out at ambient temperature at about 600×g, for about 10 mins and then 3000×g for 30 mins. Preferably the centrifugation step provides at least about 90% recovery of sperm from a semen sample.

For methods concerned with improving the viability of sperm intended for cooling and/or cryopreservation, the pellet of sperm resulting from centrifugation may be is resuspended in the presence of an increased concentration of 11β-HSD stimulator. Typically, the concentration of 11β-HSD stimulator added to the pelleted sperm equates to an amount or concentration which, when assessed at a dilution of 10% by volume, could increase 11β-HSD activity by 100% or more relative to enzyme activity measured in the absence of the stimulator.

The reconstituted sperm and hydrophobic stimulator of 11β-HSD activity may be cooled or frozen to 5° C. and below. If the sperm is intended for use in assisted conception/reproductive techniques, the semen sample may be conveniently aliquoted into individual doses sufficient to achieve fertilization. The individual dose may vary from one species to the next and is either well-known or can be readily determined.

If being cryopreserved, prior to freezing, the sperm are generally allowed to equilibrate at about 5° C. Generally, the sperm are allowed to equilibrate for a period of from 1 to about 6 hours. The preferred time is species-dependent, 3 to 6 hours being typical for pig sperm.

Typically, semen samples are collected at body temperature e.g. 37° C. for a human and around 37° C. for other mammals, and cooled to 5° C. at 0.5° C./min. Samples are then equilibrated for between 0 to 6 hours to attain resistance to cold shock. This is dependent on species where human sperm need no equilibration and pig sperm require 3 to 6 hours. Sperm is then cooled from 5° C. to −5° C. at 6° C./min in the presence of cryoprotectants such as glycerol and egg yolk. Sperm are then cooled from −5° C. to −80° C. at 40° C./min and then plunged into liquid nitrogen to a final temperature of −196° C. The optimal freezing rates will vary between species.

Promoting the Viability of Sperm that is Not Necessarily Intended for Cooling/Cryopreservation

We have also found that the use of hydrophobic stimulator fractions of the invention can improve survival rates of sperm stored at room temperature. Preferably, such storage is carried out at room temperature, e.g. from 15 to 25° C., preferably 18 to 22° C., more preferably 20° C., for a period of up to 1 day or from 1 to 7 days, for example up to 3 days, in a commercial extender diluents such as BTS (Beltsville Thawing Solution). BTS and other short-term diluents such as Beltsville Liquid (BL-1), Minois variable temperature (IVT) and Kiev are preferred for short term storage, i.e. storage for up to 1 day or from 1 to 3 days. Long-term diluents such as Acromax®, Modena, Mulberry III®, Reading, X-cell, Zorlesco and ZORPVA are preferred for longer-term storage; i.e. over 4 days.

Such storage can be carried out prior to cooling/cryopreservation or independently from it, i.e. it may be carried out without cooling and/or cryopreservation.

Methods of Obtaining a Hydrophobic Product of the Invention

The present invention also encompasses hydrophobic compounds having a stimulatory effect on 11β-HSD activity and which improves the tolerance of semen to cooling and/or cryopreservation and methods for the isolation of such compounds. A method for isolating such compounds comprises the steps of obtaining a sample of semen which is tolerant to cooling and removing the seminal plasma from the sperm by methods known in the art or described above. The seminal plasma is then separated into fractions which are enriched for the hydrophobic stimulator of 11β-HSD activity.

The Inventors have characterised fractions from boar semen which modulated the activity of 11β-HSD and which elute on a C18 column at either from 50 to 75% methanol or 95 to 100% methanol. These modulators are therefore hydrophobic and both stimulate 11β-HSD activity in semen. The C18 column which binds the modulators may be one which is available from Walters Chromatography (Hertford UK). The C18 column binds hydrophobic substances, and thus bind the modulators of the current invention since these are hydrophobic. The C18 column usually comprises silica with a pore volume of 0.5 to 1.5 ml/g, such as 0.9 to 1.15 ml/g. The column preferably has a pore diameter from 130 to 250 A e.g. from 170 to 200 A and/or a surface area of 150 to 250 m²/g e.g. from 190 to 220 m²/g. Generally the ligand bonded to such silica is octadecyldimethyl sibyl or the end capping groups are trimethyl sibyl groups.

A further embodiment of the current invention is the use of sperm whose viability is promoted by a method according to the current invention in assisted conception/reproductive techniques.

Kits for use in Determining the Suitability of a Semen Sample for Cooling and/or Cryopreservation or Storage

The present invention also provides kits for determining the suitability of a sample of semen for cooling and/or cryopreservation or storage. Such kits include at least one reagent useful for the detection of a hydrophobic stimulator of 11β-HSD activity. Suitable reagents (for direct detection or determination of hydrophobic stimulator of 11β-HSD activity/concentration) include antibodies, or suitable ligand binding agents, against the modulator. Such antibodies and reagents may be linked to a label. Typical labels are those commonly used in immunoassay procedures, for example horseradish peroxidase (HRP). Alternatively, the kit may contain antibodies, or other suitable ligand binding reagents against cortisol and/or cortisone. This may be suitable for indirect assays (measuring the level of 11β-HSD activity) such as determination of the cortisol or cortisone ratio or radiometric conversion of ³H-cortisol to ³H-cortisone. The kit may also contain standards, for example predetermined amounts of cortisone, cortisol and/or 11β-HSD, any or all of which may be labelled with a detectable label. The kit may also contain enzyme cofactors, for example, NAD⁺ or NADP⁺.

The kit may comprise agents such as oxidised tetrazolium salts to serve as a calorimetric substrate for the reoxidation of the reduced NADPH. The change in optical density of the indicator shows that the appropriate wavelength may be directly proportional to the rate of reduction of the NADP⁺ plus co-factor, which may in turn be directly proportional to 11β-HSD activity and hence proportional to the concentration of hydrophobic stimulator of 11β-HSD activity in the sample.

Assisted Reproductive/Conception Techniques

The present invention can be applied to any suitable assisted conception/reproductive technique, in particular in vitro fertilisation (IVF), artificial insemination (AI), intra-uterine insemination (MUI) and intra-cytoplasmic sperm injection (ICSI).

Therapeutics

Another aspect of the invention is a method of fertilization of oocytes in vivo using artificial insemination and/or intra-uterine insemination comprising contacting an oocyte with sperm obtained by a method of the invention under conditions which allow fertilization of the oocyte to form a zygote.

Thus, sperm obtained by a method according to the invention may be used in the manufacture of a medicament for use in an assisted conception/reproductive technique.

Another aspect of the present invention is the use of the modulators discussed above in the treatment of inflammatory disease. Two isoforms of 11β-HSD have been identified which are involved in the metabolism of cortisol to cortisone 11β-HSD2 is involved in the metabolism of active cortisol to inactive cortisone in a NAD⁺ dependent manner. 11β-HSD1 is also involved in this process but also catalyses the reverse process of the conversion of inactive cortisone to active cortisol.

It is known in the art that cortisol plays a role in the inflammatory response by directly acting to reduce inflammation. Thus, a hydrophobic stimulator of 11β-HSD1 activity identified by methods of the present invention may be used in the manufacture of a medicament for use in the treatment of inflammatory disease. The conditions of inflammation can therefore be improved by administration of such a hydrophobic stimulator of 11β-HSD activity. A therapeutically effective amount of a hydrophobic stimulator of 11β-HSD activity used in the treatment of inflammation may be given to a patient in need thereof.

In the invention, the hydrophobic stimulator of 11β-HSD activity may be used in the reduction of inflammation and may be administered in a variety of dosage forms. A preferred embodiment is to apply a formulation of the hydrophobic stimulator of 11β-HSD activity topically to a patient in need thereof in order to potentiate or lengthen the presence of naturally occurring cortisol at the site of inflammation.

The formulation of a hydrophobic stimulator of 11β-HSD activity used in the treatment of inflammation according to the invention will depend upon factors such as the nature of the exact stimulator of 11β-HSD activity.

A hydrophobic stimulator of 11β-HSD activity used in the treatment of inflammation according to the invention is typically formulated for administration with a pharmaceutically acceptable carrier or diluent. Preferably, the formulation will be a topical one. The pharmaceutical carrier or diluent may be, for example, an isotonic solution or together with the active substance, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate and, in general, non-toxic and pharmacologically inactive substances used in pharmaceutical formulations. Such pharmaceutical preparations may be manufactured in known manner, for example, by means of mixing, granulating and forming into a cream.

Suspensions and emulsions may contain as carrier, for example a natural gum, agar, sodium alginate, pectin, methylcellulose, carboxymethylcellulose, or polyvinyl alcohol. The suspensions or solutions for topical administration may contain, together with the active substance, a pharmaceutically acceptable carrier, e.g. sterile water, olive oil, ethyl oleate, glycols, e.g. propylene glycol, and if desired, a suitable amount of lidocaine hydrochloride.

A therapeutically effective amount of a substance used in the treatment of inflammation may be administered to a patient identified according to a method of the invention. The dose, for example of a hydrophobic stimulator of 11β-HSD activity, may be determined according to various parameters, especially according to the substance used; the age, weight and condition of the patient to be treated; and the required regimen. Again, a physician will be able to determine the required route of administration and dosage for any particular patient. A typical daily dose is from about 0.1 to 50 mg per kg of body weight, according to the activity of the hydrophobic stimulator of 11β-HSD activity, the age, weight and conditions of the subject to be treated and the frequency of administration. Preferably, daily dosage levels are from 5 mg to 2 g. That dose may be provided as a single dose or may be provided as multiple doses, for example taken at regular intervals, for example 2, 3 or 4 doses administered daily.

### EXAMPLES

### Example 1

Materials and Methods

Seminal Plasma and Spermatozoa Samples

Ejaculates were collected from Large White stud boars. All boars were 1-1.5 years of age of proven fertility and undergoing regular semen collection for commercial artificial inseminations. All animals received the same diet.

An entire porcine ejaculate (sperm rich fraction and the gel fraction) was manually collected (King & MacPherson, 1973) from each of 3 individual boars and shipped to the laboratory at ambient temperature within 24 hours of collection. After centrifugation of the ejaculates for 10 minutes at 600×g to sediment spermatozoa, the sperm pellet from each ejaculate was discarded. The remaining supernatant was then aspirated and centrifuged for 30 minutes at 3000×g to pellet any remaining spermatozoa and debris. The supernatant (which was still opaque) was decanted and stored at −20° C. until required for use.

The sperm rich fractions of a further 5 ejaculates, each from a different individual boar, were subsequently collected as a source of viable spermatozoa. Immediately following ejaculation, semen samples were diluted with washing buffer (447 mOsm kg⁻¹; Cat: ZS993, IMV, France) to a concentration of 100×10⁶ sperm/ml, allowed to cool from 39° C. to room temperature at approximately 0.2° C./min. The diluted semen was then shipped to the laboratory at ambient temperature within 24 hours.

Effects of Seminal Plasma on Renal 11β-HSD Activities

The effects of the porcine seminal plasma from 3 independent boars on renal 11β-HSD activities were assessed using a modification of the radiometric conversion assay for glucocorticoid oxidation previously described in our laboratory (Michael et al., 1993; Michael et al., 1995). Male Sprague-Dawley rats (200-250 g), housed in accordance with the UK Animals (Scientific Procedures) Act 1986, were allowed ad libitum access to a standard rat chow diet and drinking water. Rats were sacrificed by cervical dislocation and kidney homogenates prepared using established methods (Sewell et al., 1998; Thompson et al., 2000). Renal homogenates were utilised as a source of NADP⁺-dependent, 11β-HSD1 activity. For each assay, 1 g of rat kidney (containing approximately equal volumes of cortex and medulla) was homogenised in 18ml hypotonic Tris-EDTA lysis buffer (Rusvai & Naray-Fejes-Toth, 1993; Sewell et al., 1998; Thompson et al., 2000). After restoration of isotonicity by the addition of 2 ml 1.5M KCl (Merck, Dorset, UK), the homogenate was centrifuged at 250×g for 10 minutes (to precipitate intact tissue) and the supernatant was decanted into a fresh glass tube. From this supernatant, 100 μl volumes were transferred to glass screw-cap culture tubes, to each of which were added 600 μl of phosphate-buffered saline (PBS) (Life Technologies, Strathclyde, UK). Triplicate tubes were also prepared as assay blanks containing 100 μl of bovine serum albumen (BSA) solution (1 mg/ml prepared in PBS) in place of renal homogenate. Each triplicate set of tubes then received 100 μl seminal plasma or 100 μl PBS (controls and blanks) before being pre-incubated for 30 minutes at 37° C. in a gyratory waterbath.

To initiate the 11β-HSD assay, each tube received 100 μl NADP+(4 mM in PBS)(Sigma, Dorset, UK) and 100 μl PBS containing 0.5 μCi [1,2,6,7-³H]-cortisol (Amersham, Aylesbury, Bucks, UK) plus unlabelled cortisol (Sigma, UK) (to a final steroid concentration of 100 nM). Tubes were then returned to the waterbath for 60 minutes, after which reactions were terminated by the addition to each tube of 2 mls ice-cold chloroform (Merck, UK). To partition the organic and aqueous phases, these tubes were centrifuged at 1000×g for 30 minutes at 4° C. After aspirating the aqueous supernatant, the organic extracts were evaporated to dryness under nitrogen at 60° C. The steroid residues were resuspended in 20 μl ethyl acetate containing 1 mM cortisol and 1 mM cortisone (Sigma, UK), and were resolved by thin layer chromatography (TLC) using Silica 60 TLC plates (Merck, UK) in an atmosphere of 92:8 (v/v) chloroform: 95% (v/v) ethanol (Merck, UK). After quantifying [³H]-cortisol and [³ H]-cortisone using a Bioscan 200 TLC radiochromatogramme scanner (LabLogic, Sheffield, UK), 11β-HSD activities were calculated as μmol cortisol oxidised to cortisone over 60 minutes and standardised per mg protein in the renal homogenate, where protein concentrations were measured using the Biorad protein assay (Bradford et al., 1976; Rosa et al., 1980).

Fractionation of Seminal Plasma by C18 Column Chromatography

Each of the 3 samples of seminal plasma tested in the assays described above were subsequently fractionated using the method previously described by Thurston et al (2002a). Aliquots (1 ml) of independent seminal plasma samples were applied to separate C18 Sepak cartridges (Amersham, UK) that had previously been conditioned with 20 ml methanol and washed with 20 ml double-distilled water (DDW). After collecting the loading eluent (i.e. that fraction of the sample not retained by the column), the column was sequentially eluted with lml volumes of a stepwise gradient of 0-100% (v/v) methanol (Merck, UK) in DDW. All 1 ml fractions were collected into borosilicate tubes and those samples eluted at methanol concentrations greater than 20% (v/v) methanol were evaporated to dryness under nitrogen before being resuspended in lml volumes of 20% (v/v) methanol in DDW. Parallel samples of DDW and PBS only were similarly fractionated as negative controls.

Effects of Seminal Plasma Fractions on Renal 11β-HSD Activities

Assays of renal NADP⁺-dependent 11β-HSD activities were performed as described above with the following modification. Samples were incubated in triplicate in the presence of (a) 100 μl of a specific seminal plasma fraction, or (b) 100 μl of 20% (v/v) methanol in DDW (i.e. final methanol concentration in 1 ml=2%) or (c) 100 μl of DDW alone. Enzyme activities in the presence of the 0% and 10% (v/v) methanol fractions were compared to those measured in the controls incubated with DDW alone, whereas enzyme activities in the presence of fractions eluted at ≧20% (v/v) methanol were compared to the 20% (v/v) methanol control.

Spermatozoa Cooling Protocol

As noted above, following collection, semen was diluted into a washing buffer (447 mOsm kg-1; Cat: ZS993, IMV, France) to a final concentration of 100×10⁶ spermatozoa/ml, and allowed to cool from 39° C. to room temperature at approximately 0.2° C./min. Semen was then further diluted to a concentration of 2×10⁶ spermatozoa/ml with Tyrode's Complete Media (Tyrode's Albumin Lactate Pyruvate; TALP; Parrish et al., 1988) and semen quality was assessed before cooling as detailed below. A total of lml of diluted semen (900 μl semen plus 100 μl seminal plasma or 100 μl lipid extract generated the method described below) was cooled at a linear rate from 23° C. to 5° C. over 100 minutes at a rate of 0.18° C./min using a GP200 waterbath controller (Grant, Cambridge, UK). On reaching 5° C., cooled samples were immediately re-warmed by incubation at 39° C. for 10 minutes.

Sperm Cryopreservation Experiments

Following ejaculation, semen was diluted into Beltsville Thawing Solution (BTS; 37 g/L glucose monohydrate anhydrous, 6 g/L sodium citrate, 1.25 g/L sodium hydrogen carbonate, 1.25 g/L EDTA-disodium, 0.75 g/L KCl, pH 7.2) to a final concentration of 1×10⁸ sperm per ml and allowed to cool from 39° C. to room temperature. Fifty millilitres of semen (1×10⁸/ml) was centrifuged at 15° C. for 15 min, at 500 g and the supernatant containing seminal plasma removed. The sperm pellet was subsequently used for cryopreservation experiments.

For cryopreservation experiments, the sperm pellet was diluted to a concentration of 2×10⁶ spermatozoa/ml in a commercial preservation diluent (Boarciphos: IMV, L'Aigle, France) supplemented with 20% (v/v) seminal plasma fraction (produced by C18 column chromatography as described previously). Diluted sperm was cooled from 15° C. to 5° C. at approximately 0.2° C./min then cryopreserved using a Planer Products Kryo Save Compact KS1.7/Kryo 10 Control freezing system. Sperm were cooled from 5° C. to −5° C. at 6° C./min, further cooled from −5° C. to −80° C. at 40° C./min and finally cooled at 70° C./min to −150° C. and plunged into liquid nitrogen (Thurston et al (2002b)). Sperm samples were stored in liquid nitrogen for 24 hours, thawed by plunging into a 40° C. water bath for 1 min and assessed for sperm survival.

Spermatozoa Viability Assessment

Each semen sample was assessed for sperm quality before and after cooling. Plasma membrane integrity was used as an assessment of sperm viability. Semen was diluted to a concentration of 2×10⁶ spermatozoa/ml with TALP and stained with the fluorescent probes SYBR-14 and propidium iodide according to the manufacturer's instructions (Live/Dead Sperm Viability Kit; Molecular Probes Europe, Leiden, The Netherlands)(Gamer & Johnson, 1995). Plasma membrane integrity assessments were carried on a Coulter Epics XL instrument flow cytometer (Coulter Corp. Miami, Fla., USA). Flow cytometery set up conditions for SYBR-14 and PI have been validated previously (edrano, 1998). Three flow cytometry readings were taken from each thawed straw.

Effects of Seminal Plasma on Spermatozoa Viability Following Cooling

The effects of the porcine seminal plasma from the 3 independent boars on sperm viability following cooling and re-warming was investigated. Sperm cooling studies and viability assessments were performed as described above. Spermatozoa, isolated from 5 different animals, were cooled and re-warned in triplicate in the presence of (a) 100 μl seminal plasma from the 3 test boars, or (b) 100 μl TALP only. Sperm viability assessments for semen cooled in the presence of the test seminal plasma samples were compared to those measured in control aliquots of semen incubated with TALP medium alone.

Extraction of Lipids from Seminal Plasma

The lipid components of each of the 3 samples of seminal plasma tested in the cooling assays described above were concentrated by extraction into chloroform:methanol as previously described (Eng & Oliphant, 1978). Aliquots (1 ml) of the seminal plasma samples were mixed with 5 ml of 2:1 (v/v) chloroform:methanol by vortexing for approximately 15 seconds. After centrifugation for 30 minutes at 2000×g at 4° C., the aqueous layer of each sample was decanted and subjected to two more cycles of chloroform:methanol extraction. Lipids removed from the 3 chloroform phases of the extraction mixture were combined and evaporated to dryness under nitrogen before being resuspended in lml volumes of TALP, acetone, DMSO or ethanol (since the solubility of the extracted lipids was unknown). Lipid extracts were stored at −20° C. until use.

Effects of Seminal Plasma Lipid Fractions on Spermatozoa Viability following Cooling

Sperm cooling studies and viability assessments were performed as described above with the following modification. Spermatozoa isolated from the ejaculates of 5 independent boars were cooled and re-warmed in triplicate in the presence of (a) 100 μl of the test seminal plasma lipid extracts reconstituted in TALP/acetone/DMSO/ethanol, or (b) 100 μl TALP/acetone/DMSO/ethanol without seminal lipids. Sperm viability assessments made in semen samples cooled in the presence of the seminal plasma lipid extracts were compared to those measured in the controls cooled in the presence of the relative solvent minus seminal lipids.

Results/Conclusions

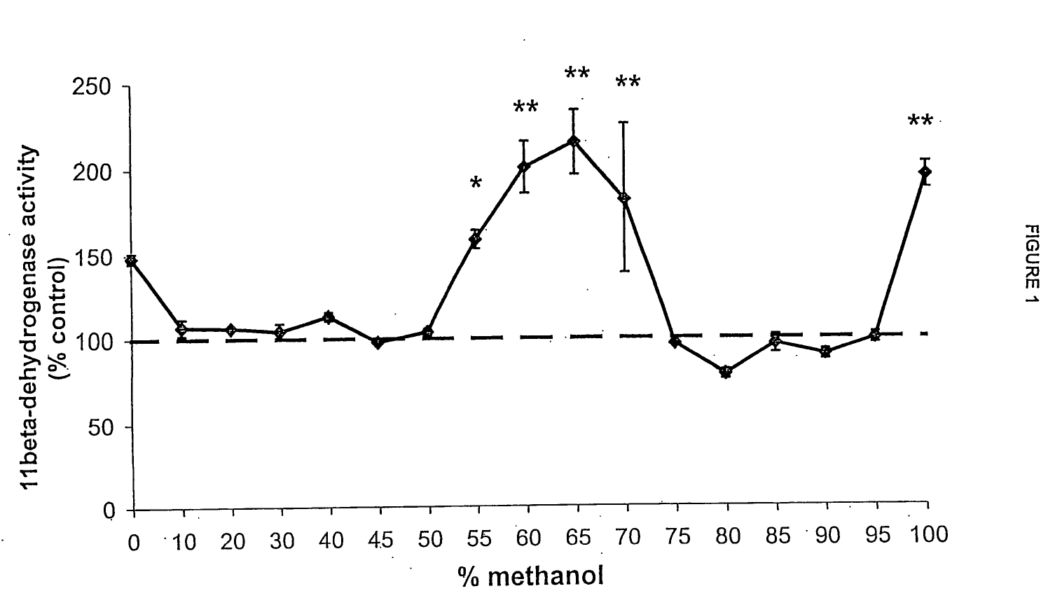

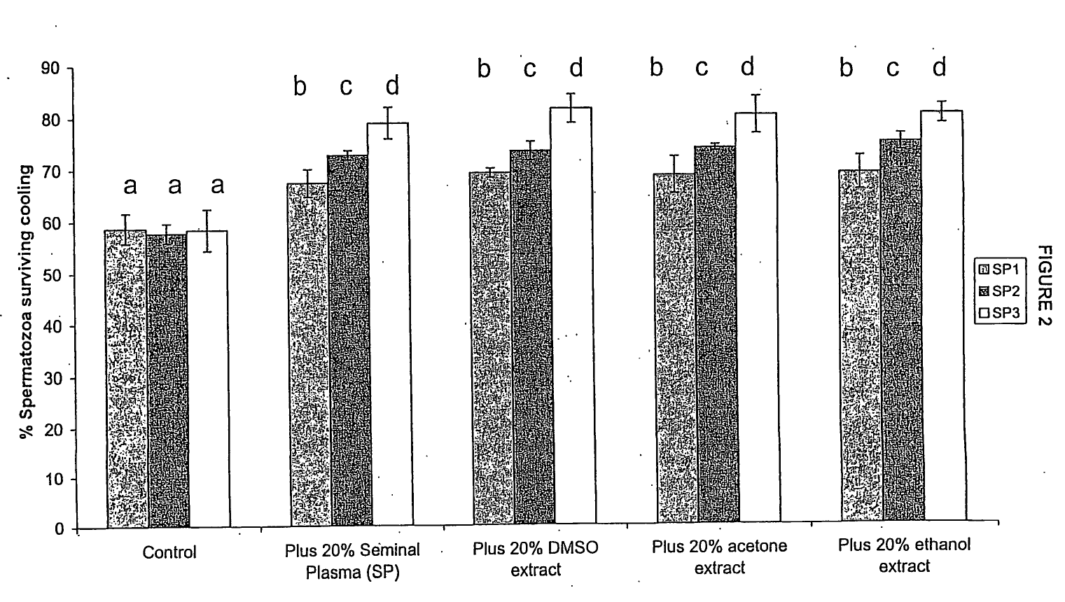

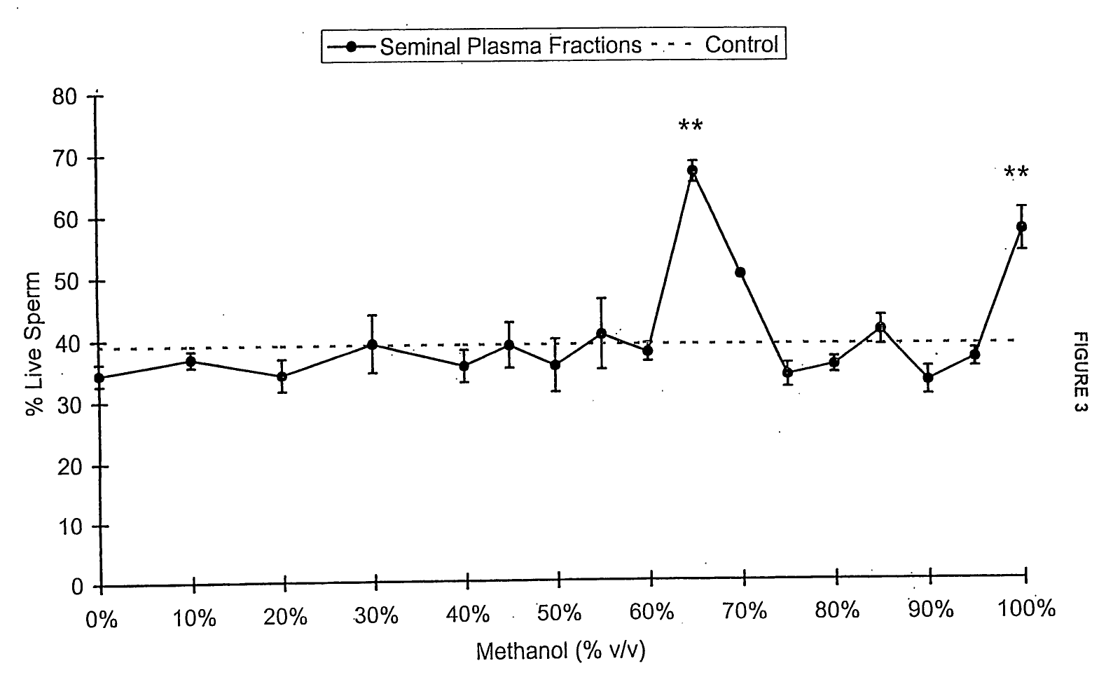

1. 3 samples of boar seminal plasma (prior to C18 fractionation) stimulated NADP⁺-dependent oxidation of glucocorticoids by 11β-HSD1 within 1 hour in rat kidney homogenates by 68, 76 and 85% (relative to controls).
2. Using C18 column chromatography, it was possible to resolve hydrophobic compounds from seminal plasma that could account for the acute stimulation of NADP⁺-dependent glucocorticoid oxidation by 11β-HSD1 (FIG. 1).
3. The major stimulatory fractions of seminal plasma were resolved at methanol concentrations between 55-75% (v/v) methanol, with a second (minor) stimulatory activity eluted at 100% (v/v) methanol (FIG. 1).
4. The degree of stimulation exerted by those hydrophobic fractions of seminal plasma eluted across the range of 55-75% (v/v) methanol varied between seminal plasma samples obtained from different boars (range=232% to 504% stimulation).
5. The degree of stimulation exerted by those hydrophobic fractions of seminal plasma eluted across the range of 55-75% (v/v) methanol did not correlate with the stimulation of 11β-HSD1 activity by the corresponding seminal plasma samples prior to C18 column chromatography (Table 1).
6. When the samples of seminal plasma were added to spermatozoa (from different boars) to protect against cooling-induced cell death, the ability of different seminal plasma samples to reduce the risk of cell death increased with their content of hydrophobic stimulator(s) of 11β-HSD1 (Table 1).
7. The protective effects of the tested seminal plasma samples were reproduced precisely (in terms of degree of cell protection) by lipid extracts of the seminal plasma extracted into 3 different organic solvents (ethanol, acetone and dimethylsulphoxide) (FIG. 2).
8. Taking observations 6 and 7 together implicates the hydrophobic stimulator(s) of 11β-HSD1 as the active hydrophobic components of seminal plasma that protect sperm from cooling-induced death.

9. Addition of the hydrophobic fraction of seminal plasma shown previously to stimulate 11β-HSD1 activity significantly increased the survival of sperm following cooling, cryopreservation and thawing to room temperature (FIG. 3).

TABLE 1

| | Stimulation of 11 β-HSD1 activity | | | Effect on |
|---|---|---|---|---|
| | (percentage increase relative to | | | cooling- |
| | control enzyme activity) by: | | | induced sperm |
| Seminal | | Hydrophobic | Hydrophobic | death |
| Plasma | SP prior to | SP components | SP components | (percentage of |
| Sample | fraction- | eluted at | eluted at | spermatozoa |
| No. | ation | 55-70% methanol | 100% methanol | surviving) |
| 1 | 76 ± 6 | 232 ± 13 | 85 ± 6 | 67.5 ± 2.7 |
| 2 | 85 ± 5 | 341 ± 14 | 111 ± 5 | 72.9 ± 0.9 |
| 3 | 68 ± 6 | 504 ± 13 | 94 ± 2 | 79.1 ± 3.1 |

### Example 2

Resolution of Enzyme Modulators from Bovine and Porcine Seminal Plasma Using C18 Column Chromatography, Assessment of Resulting Fractions for Effects on 11βHSD Activities Using Rat Kidney Homogenate Bioassay and Correlation of Indices of Semen Quality of Ejaculates with the Levels of the Modulators of 11βHSD1 Activity

Enzyme modulators have been resolved and assessed from 10 bovine and 8 porcine seminal plasma samples.

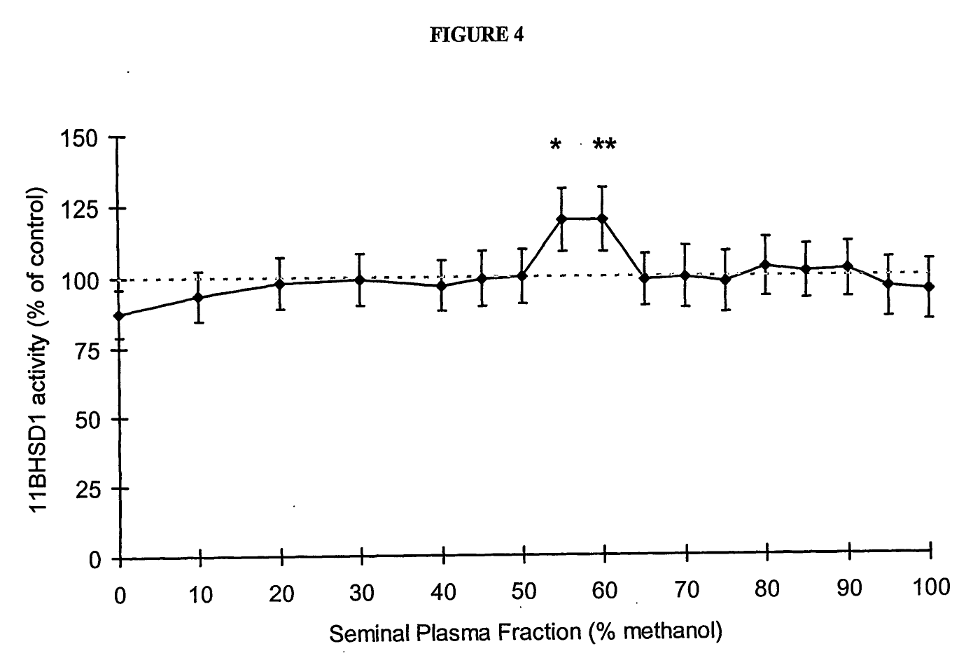

In the samples of bovine seminal plasma, the main stimuli of 11βHSD1 activity elute in those hydrophobic fractions eluted at 55% and 60% (v/v) methanol (FIG. 4).

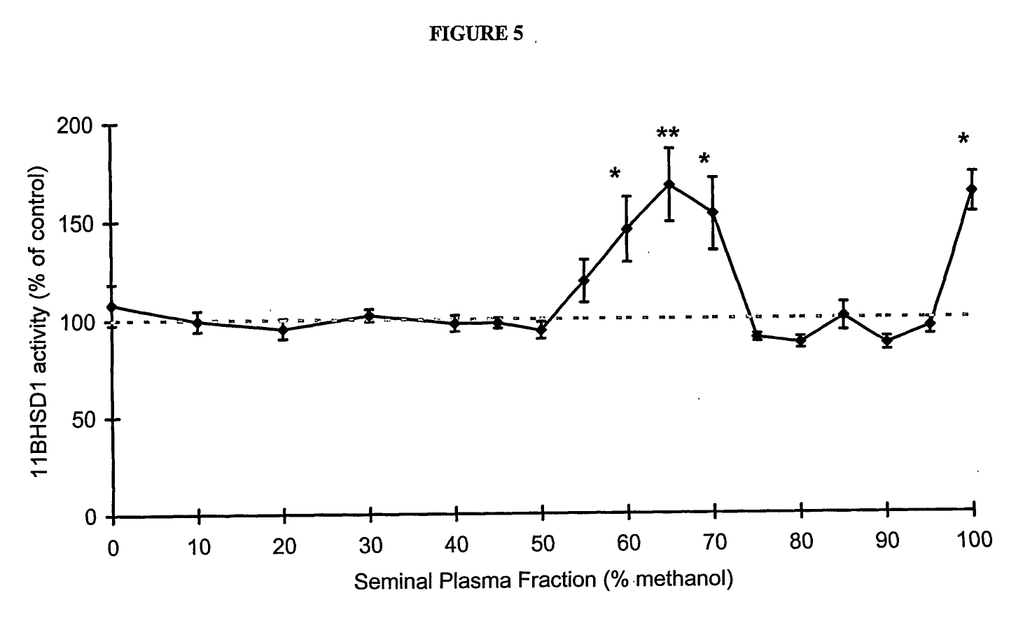

In the samples of porcine seminal plasma, the main stimuli of 11βHSD1 activity elute in those fractions eluted between 55% and 70% (v/v) methanol with a secondary stimulus eluted at 100% (v/v) methanol (FIG. 5).

With the active hydrophobic fractions of bull semen there was a 0-50% increase in 11βHSD1 activity in the presence of the bull semen fraction eluted at 55% (v/v) methanol relative to control enzyme activity. With the corresponding fraction of boar semen eluted at 65% (v/v) methanol, there was a 0-144% increase.

Notwithstanding the differences in the level of enzyme stimulation exerted by seminal plasma from the 2 species, correlation analyses have been completed for 6 bull and 6 boar seminal plasma samples.

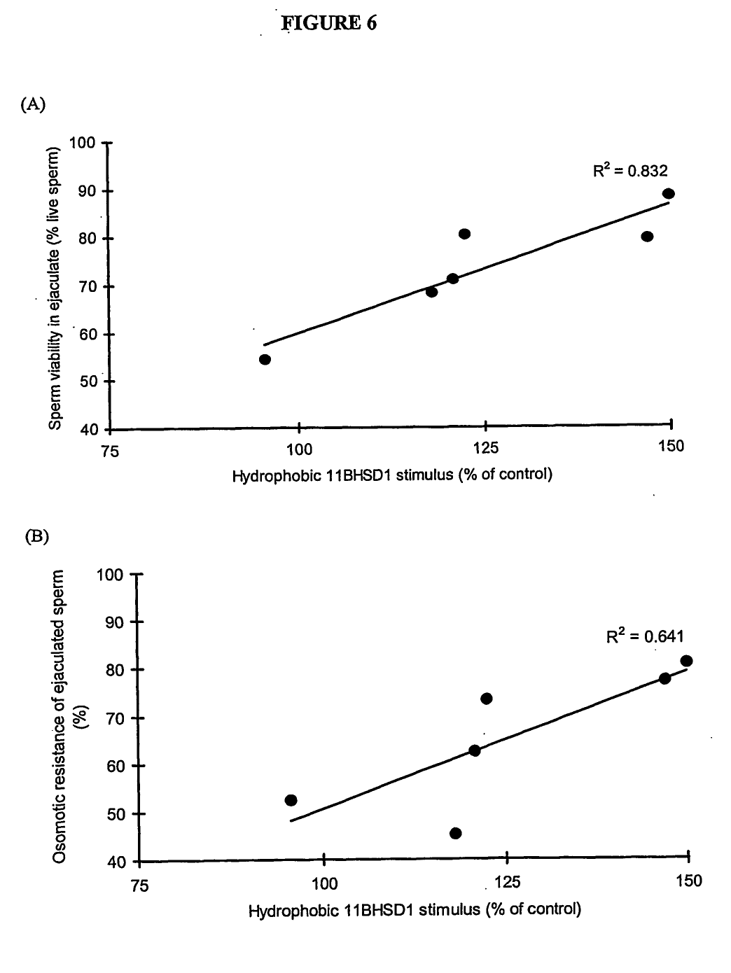

Indices of bull semen quality correlated with the stimulation of 11βHSD1 activity by the fraction of seminal plasma eluted at 55% (v/v) methanol. Specifically, the proportion of live sperm in each ejaculate and the osmotic resistance of those sperm each increased with the level of 11βHSD1 stimulation (R²=0.832 and 0.641, respectively; FIG. 6). These correlations were not particularly strong, possibly reflecting the low levels of enzyme stimulation achieved with the hydrophobic fractions of bull semen.

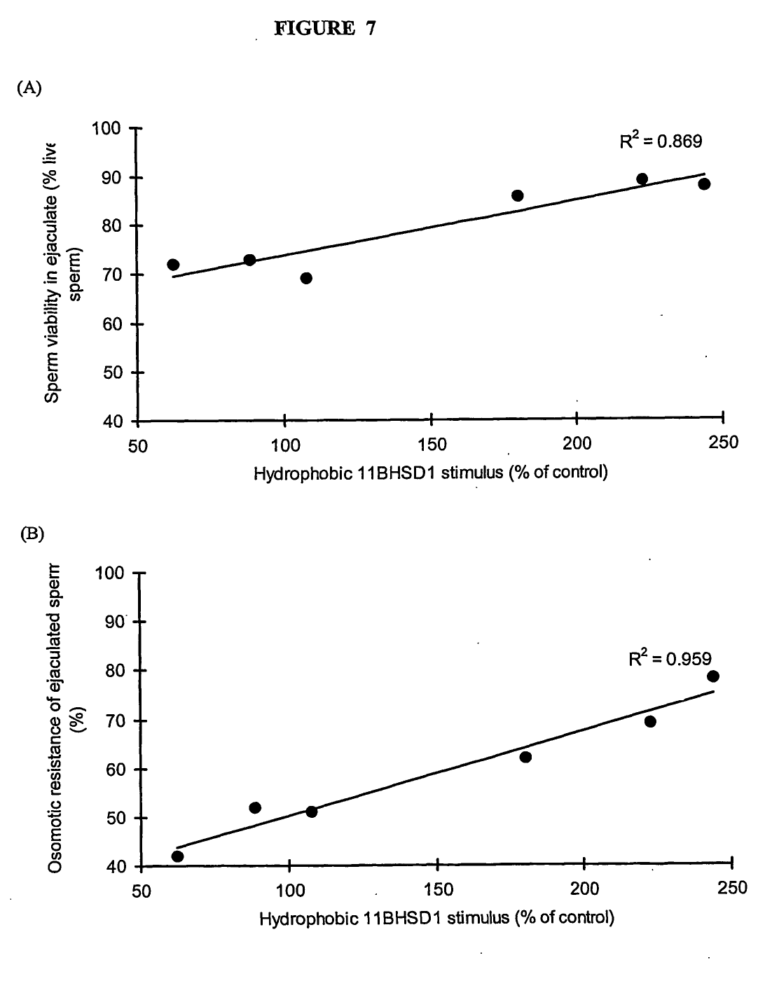

Indices of boar semen quality correlated more strongly with the stimulation of 11βHSD1 activity by the fraction of boar seminal plasma eluted at 65% (v/v) methanol. Both the proportion of live sperm in each ejaculate and the osmotic resistance of those sperm increased with the level of 11βHSD1 stimulation (R²=0.869 & 0.959, respectively; FIG. 7).

Among 6 boar seminal plasma samples, 3 contained no hydrophobic stimulus to 11βHSD 1. The proportion of live sperm in these ejaculates (mean±SD=71±2%) was significantly lower than in 3 ejaculates which contained high levels of hydrophobic stimulus to 11βHSD1 (88±2% viability). Likewise, the osmotic resistance of sperm from the 3 ejaculates that lacked detectable stimulus to 11βHSD1 was significantly lower than the osmotic resistance of sperm from those 3 ejaculates which contained high levels of hydrophobic stimulus to 11βHSD1 (48±6% versus 70±8%, respectively).

Determination of the Effects of the 11βHSD Stimulatory Fractions on Sperm Survival in a Commercial Extender Diluent at Room Temperature

After 3 days storage in a commercial extender diluent (BTS), the average survival of control bull sperm (stored in the absence of fractions of seminal plasma) was 83±1% viability. Three bull semen samples were tested for effects on bull sperm survival for 72 hours at room temperature, where unfractionated (whole) seminal plasma or specific fractions of bull seminal plasma were added on the second day at room temperature.

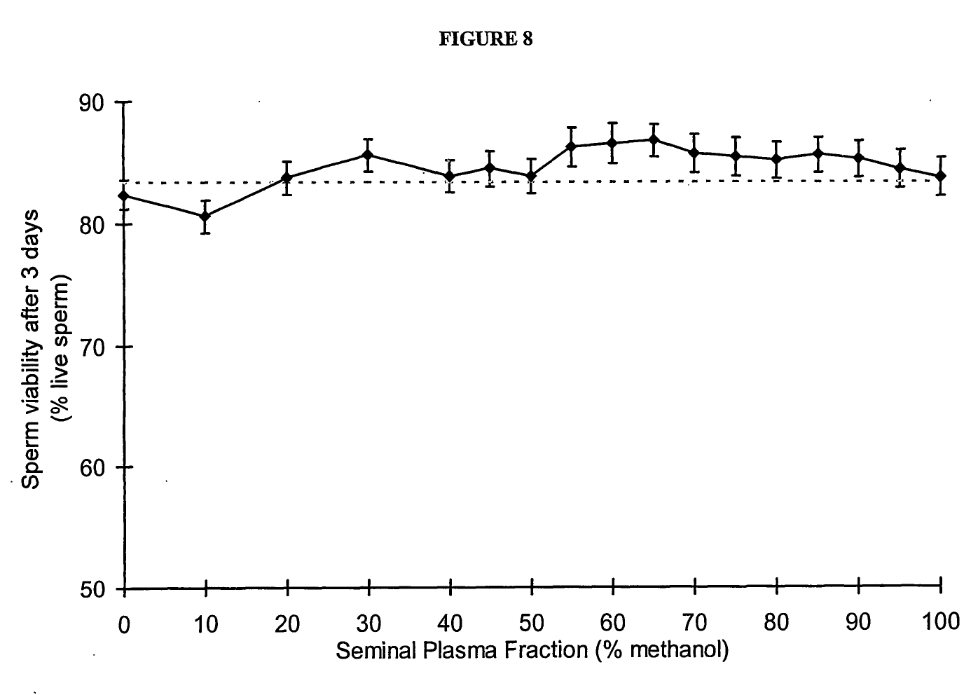

Addition of whole bovine seminal plasma had no significant effect on sperm survival at 72 hours (84±3% viability). Addition of the hydrophobic fractions of bovine seminal plasma, eluted at 55% to 65% (v/v) methanol, increased sperm survival up to 87±1% viability (FIG. 8).

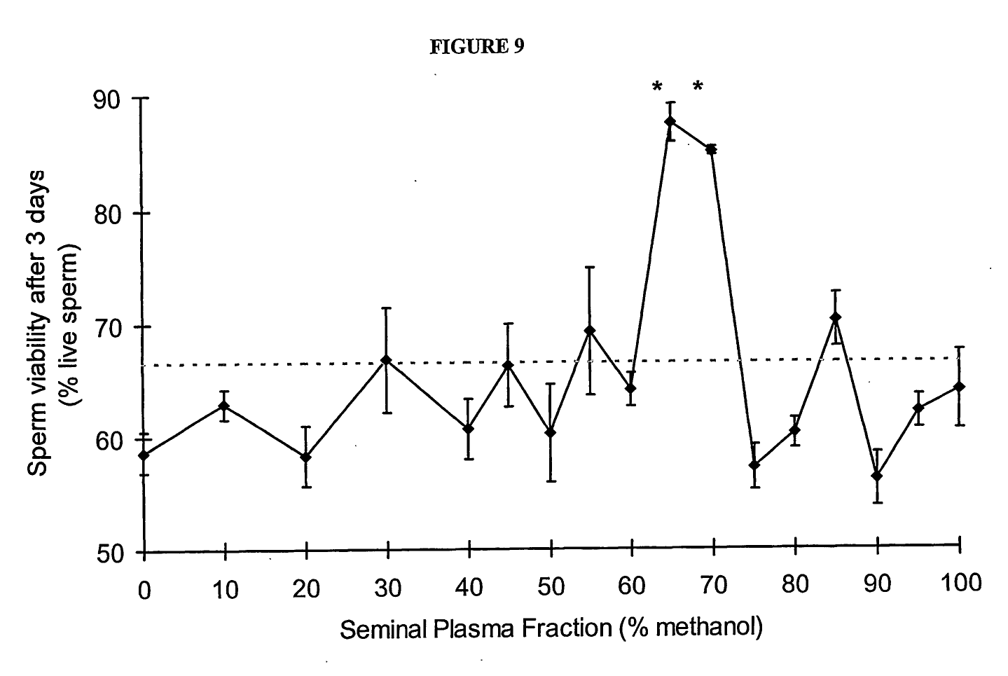

After 3 days storage in a commercial extender diluent, the average survival of control boar sperm (stored in the absence of fractions of seminal plasma) was 67% viability. Addition of whole boar seminal plasma increased sperm survival at 72 hours to 88% viability. Addition of the hydrophobic fractions of boar seminal plasma, eluted at 65% and 70% (v/v) methanol, increased sperm survival up to 88% and 85% viability, respectively (FIG. 9).

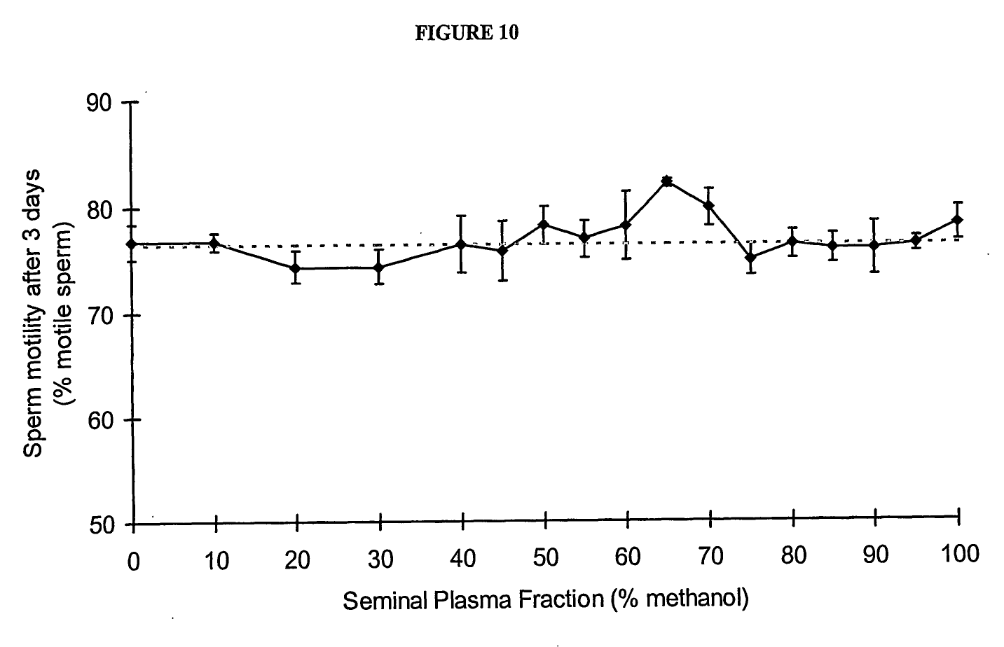

The average motility of boar sperm following 3 days storage at room temperature in BTS extender diluent was 76±2% motility (n=3). Addition of whole boar seminal plasma increased sperm motility at 72 hours to 80±1% motile sperm. Addition of the hydrophobic fractions of boar seminal plasma, eluted at 65% and 70% (v/v) methanol, increased sperm motility up to 82±1% and 80±3% motility, respectively (FIG. 10).

Determination of the Effects of the 11βHSD Stimulatory Fractions on Sperm Survival During Cooling and/or Cryopreservation

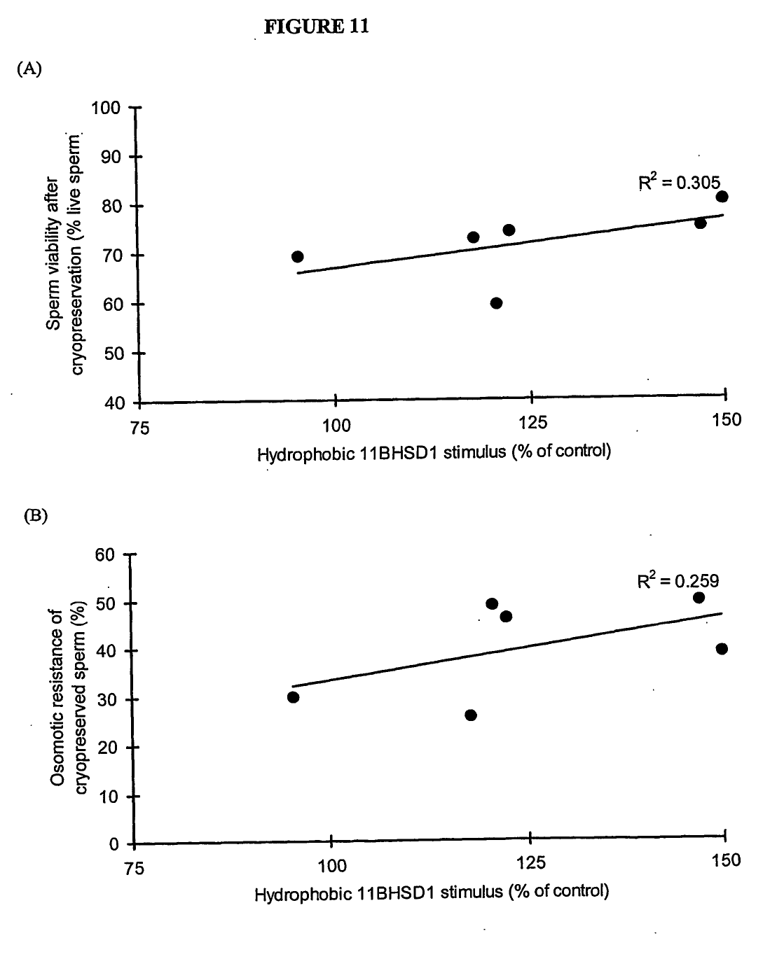

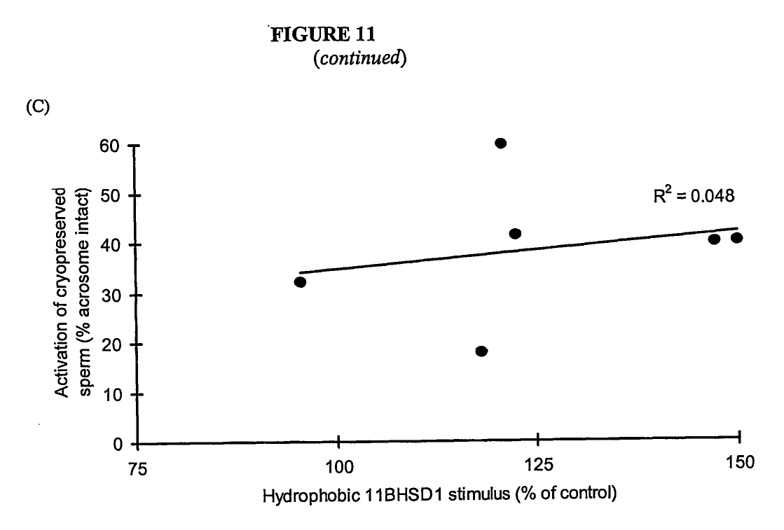

Indices of bull semen quality following cryopreservation and thawing correlated, albeit weakly, with the stimulation of 11βHSD1 activity by the fraction of bull seminal plasma eluted at 55% (v/v) methanol. Specifically, the proportion of live sperm in each ejaculate and the osmotic resistance of those sperm post-thaw tended to increase with the level of 11βHSD1 stimulation (R²=0.305 & 0.259, respectively; FIG. 11). However, there was no correlation in bull semen with the proportions of sperm retaining intact acrosomes after cryopreservation and thawing (FIG. 11C).

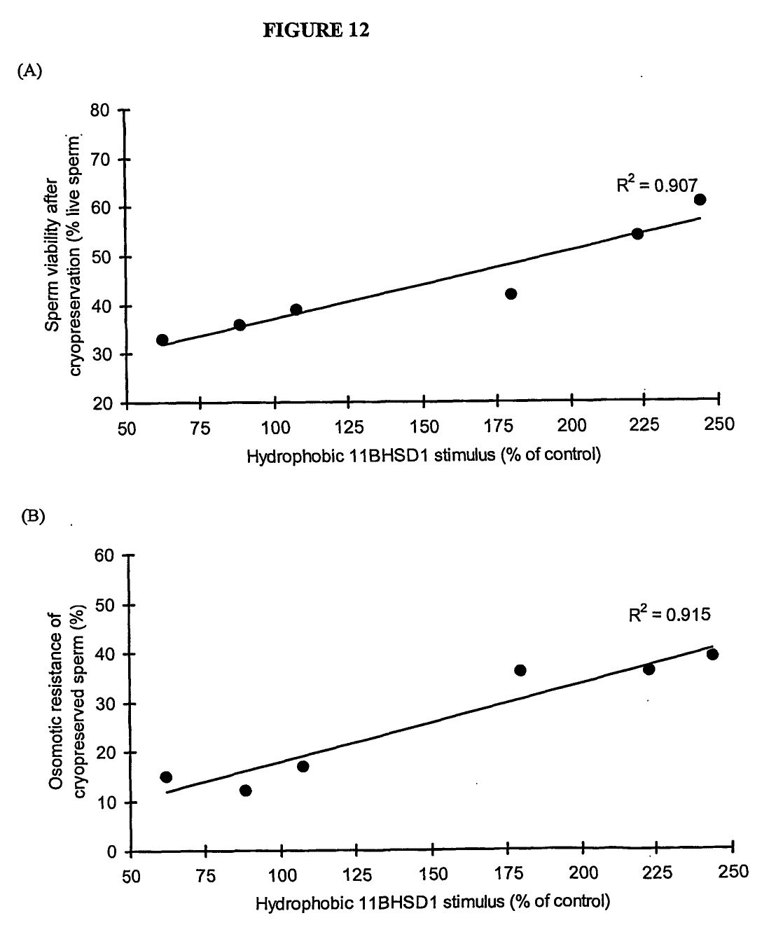

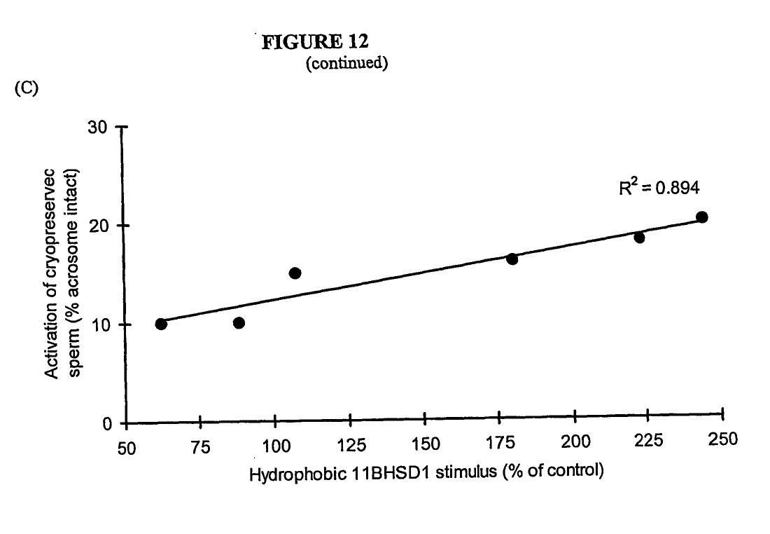

Indices of boar semen quality following cryopreservation and thawing correlated more strongly with the stimulation of 11βHSD1 activity by the fraction of boar seminal plasma eluted at 65% (v/v) methanol. Both the proportion of live sperm in each ejaculate and the osmotic resistance of those sperm post-thaw increased with the level of 11βHSD1 stimulation (R²=0.907 & 0.915, respectively, FIG. 12). In addition, the resistance of sperm to hyperactivation (as assessed by the proportion of sperm with remaining “acrosome intact” after cryopreservation) also increased with the level of 11βHSD1 stimulation by the hydrophobic fractions of the corresponding seminal plasma samples (R²=0.894; FIG. 12C).

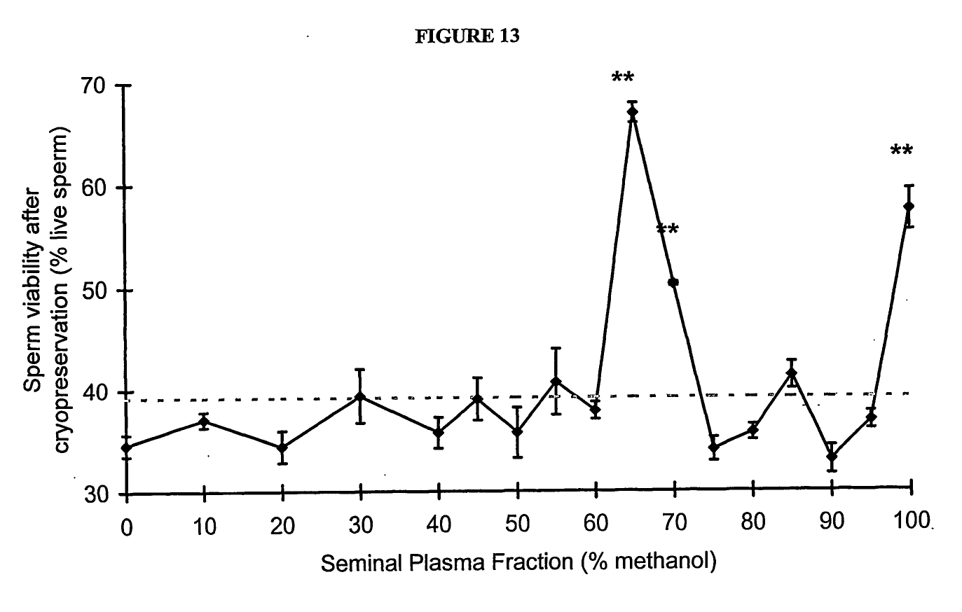

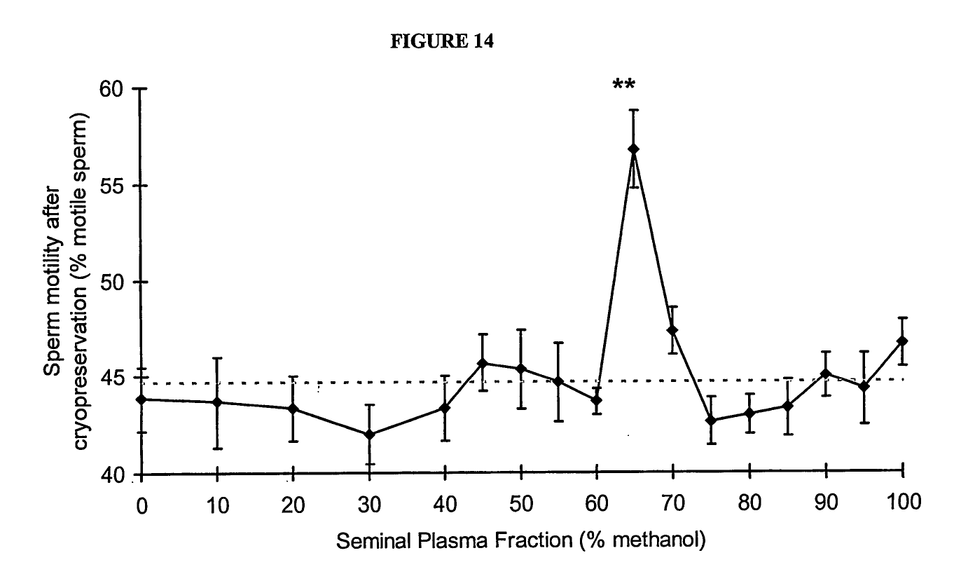

Following cryopreservation and thawing in the absence of seminal plasma, the proportion of boar sperm surviving was 39%. However, sperm survival was increased to between 50 and 70% by inclusion of the hydrophobic fractions of boar seminal plasma (eluted at 65%, 70% and 100% (v/v) methanol) during cryopreservation (FIG. 13). Following cryopreservation and thawing in the absence of seminal plasma, the proportion of boar sperm retaining motility was 45%. However, sperm motility was increased to 57% by inclusion of the hydrophobic fraction of boar seminal plasma (eluted at 65% (v/v) methanol) during cryopreservation (FIG. 14).

### REFERENCES

Bradford M M (1976) A rapid and sensitive method for the quantitation of microgram quantities of protein utilising the principle of protein dye binding. Analytical Biochemistry 72: 248-254
Eng L A and Oliphant G (1978) Rabbit sperm reversible decapacitation by membrane destabilisation with a highly purified glycoprotein from seminal plasma Biology of Reproduction 19: 1083-1094
Garner D L and Johnson L A (1995) Viability assessment of mammalian spermatozoa using SYBR-14 and propidium iodide Biology of Reproduction 53:276-284
King G J and MacPherson J W (1973) A comparison of two methods for boar semen collection. Journal of Animal Science 36: 563-565
Medrano H J A (1998) The importance of individual variation in boar semen cryopreservation. PhD Thesis, Veterinary Basic Sciences, Royal Veterinary College, University of London, UK.
Michael A E, Gregory L, Walker S M, Antoniw J W, Shaw R W, Edwards C R W and Cooke B A (1993) Ovarian 11β-HSD: potential predictor of conception by in-vitro fertilization and embryo transfer. Lancet 342: 711-712
Michael A E, Gregory L, Piercy E C, Walker S M, Shaw R W, Cooke B A (1995) Ovarian 11β-HSD activity is inversely related to the outcome of in vitro fertilization-embryo transfer cycles. Fertil. Steril 64(3): 590-8
Nacharaju V L, Muneyyirci-Delale O and Khan N (1997) Presence of 11β-HSD in human semen: evidence of correlation with semen characteristics. Steroids 62: 311-314
Parrish J J et al (1998) Capacitation of bovine sperm by heparin. Biology of Reproduction. 38: 1171-1180
Rosa J, Senan C, Boluquit Y, Garel M C and Molko F (1980) Evaluation of 3 commercial kits for the estimation of total glycolysed haemaglobin. Annales de Biologie Clinique (Paris) 38:183-189
Rusvai E and Naray-Fejes-Toth A. A new isoform of 11β-HSD in aldosterone target cells Journal of Biological Chemistry 268:10717-10720
Sewell K J, Shirley D G, Michael A E, Thompson A, Norgate D P and Unwin R J (1998) Inhibition of renal 11β-HSD in vivo by carbenoxolone in the rat and its relationship to sodium excretion Clinical Science 95: 435-443
Thurston L M, Norgate D P, Jonas K C, Chandras C, Kloosterboer H J, Cooke B A and Michael A E (2002a) Ovarian modulators of 11β-HSD in follicular fluid from gonadotrophin-stimulated assisted conception cycles. Reproduction 124: 801-812
Thurston L M, Mileham A, Siggins K, Watson P F and Holt W V (2002b) Identification of amplified restriction fragment length polymorphism (AFLP) markers linked to genes correlated with boar sperm quality following cryopreservation. Biology of Reproduction 66: 545-554
Thurston L M, Norgate D P, Jonas K C, Gregory L, Wood P J, Cooke B A and Michael A E (2003a) Ovarian modulators of 11β-HSD1 activity and intra-follicular cortisol:cortisone ratios correlate with the clinical outcome of in vitro fertilization-embryo transfer (IVF-ET). Human Reproduction 18: 1603-1612
Thurston L M, Jonas K C, Abayasekara D R E and Michael A E (2003b) Ovarian modulators of 11β-HSD activity in follicular fluid from bovine and porcine large antral follicles and spontaneous ovarian cysts. Biology of Reproduction 68: 12157-2163

1-34. (canceled)

35. A method of determining the suitability of a sample of mammalian semen for cooling and/or cryopreservation or storage, said method comprising:
(a) providing said sample of semen;
(b) determining the level of a hydrophobic stimulator of 11β-HSD activity in said sample; and
(c) assessing, from the level of 11β-HSD stimulator determined, the suitability of the semen sample for cooling and/or cryopreservation or storage.

36. A method according to claim 35 wherein said sample of semen is from a human male.

37. A method according to claim 35 wherein said sample of semen is of rodent, bovine, equine, porcine or ovine origin.

38. A method according to claim 35 wherein said hydrophobic stimulator of 11β-HSD activity elutes in a fraction from a C18 column at either 50 to 75% or 95 to 100% methanol.

39. A method according to claim 35 wherein said determination of the level of hydrophobic stimulator of 11β-HSD activity is by contacting said sample of semen with 11β-HSD present in another body fluid or another body derived substance, and determining the effect of the hydrophobic stimulator on the activity of 11β-HSD.

40. A method according to claim 39 wherein said contacting is performed by adding 11β-HSD and a substrate of 11β-HSD to said semen sample.

41. A method according to claim 40 wherein said substrate is ³H-cortisol or ³H-corticosterone.

42. A method according to claim 35 wherein said other body derived substance is a homogenised animal organ.

43. A method according to claim 42 wherein said animal organ is an animal kidney.

44. A method according to claim 43 wherein said animal organ is a rodent organ.

45. A method according to claim 44 wherein said rodent organ is a rat organ.

46. A method according to claim 45 wherein said rat organ is a rat kidney.

47. A method according to claim 35 wherein a control assay is conducted to allow for any 11β-HSD already present in said sample from said male individual.

48. A method of improving the survival rate of sperm or promoting the viability of sperm, said method comprising:
(a) providing a sample of semen; and
(b) combining said sample of semen with an increased concentration of a hydrophobic stimulator of 11β-HSD activity; and optionally
(c) storing said combination of semen and hydrophobic stimulator for a period of time.

49. A method according to claim 48 wherein said hydrophobic stimulator of 11β-HSD activity elutes in a fraction from a C18 column at either 50 to 75% or 95 to 100% methanol.

50. A method according to claim 48 wherein said increased concentration of hydrophobic stimulator of 11β-HSD activity is an amount or concentration of 11β-HSD stimulator which, when assessed at a dilution of 10% by volume, could increase 11β-HSD activity by 100% or more relative to enzyme activity measured in the absence of the stimulator.

51. A method according to claim 48 further comprising
(a) a step of cooling said combination of semen and hydrophobic stimulator of 11β-HSD activity to 5° C. or below; and/or
(b) a step of freezing said combination of sperm and hydrophobic stimulator of 11β-HSD activity.

52. A method according to claim 48 wherein sperm is removed from said sample of semen and said sperm is combined with an increased concentration of a hydrophobic stimulator of 11β-HSD activity.

53. A method according to claim 48 wherein said combination of semen and hydrophobic stimulator is stored without cooling or cryopreservation.

54. A method according to claim 48 wherein 85% or more of said human sperm, 40% of said pig sperm, 50% of said horse sperm, 70% of said cow sperm, 50% said sheep sperm or 60% of said rodent sperm survive said cooling and/or cryopreservation or said storage.

55. A method of fertilizing an oocyte in vitro comprising contacting said oocyte with sperm obtained by a method according to claim 48.

56. A method of performing an assisted conception/reproductive procedure comprising contacting an oocyte with sperm obtained by a method according to claim 48 under conditions which allow fertilization of the oocyte.

57. A method according to claim 56 wherein said assisted conception/productive procedure is an IVF procedure comprising contacting said oocyte and said sperm in vitro and introducing the fertilized oocyte or zygote or embryo derived therefrom into a female such that it may develop to term.

58. A method according to claim 56 wherein said assisted conception/reproductive procedure is an artificial insemination (Al) procedure.

59. A method according to claim 58 wherein said artificial insemination is an intra-uterine insemination (IUI) procedure.

60. A method according to claim 56 wherein said assisted conception/reproductive procedure is an intracytoplasmic sperm injection (ICSI) procedure.

61. A method of obtaining a hydrophobic product that improves the tolerance of mammalian semen to cooling and/or cryopreservation or storage, comprising the steps of:
(a) providing a sample of semen;
(b) removing the seminal plasma from the sperm; and
(c) fractionating the seminal plasma of (b) to enrich for said product.

62. A method according to claim 61 wherein said seminal plasma is removed from said sperm by centrifugation, Percoll centrifugation or Percoll swim-up.

63. A method according to claim 61 wherein said fractionating of said seminal plasma is on a C18-methanol affinity chromatography column, TLC, HPLC or FPLC.

64. A product obtainable by fractionation of mammalian seminal plasma and having a stimulatory effect on 11β-HSD activity, which improves the tolerance of semen to cooling and/or cryopreservation or storage.

65. A product according to claim 64 which is obtainable by a method cited above.

66. Use of a product of claim 64 to improve the tolerance of semen to cooling and/or cryopreservation or storage.

67. A method of treatment of inflammatory disease by administering an amount of a product of claim 64 effective to increase the survival of topically applied cortisol or cortisol already circulating within the bloodstream.

68. A method of treatment of inflammatory disease by administering an amount of a product of claim 64 effective to stimulate the production of cortisol from circulating cortisone by stimulation of 11β-HSD1.
